(19) United States
(12) Patent Application Publication
(10) Pub. No.: US 2006/0281071 A1
(43) Pub. Date: Dec. 14, 2006

(54) Stearoyl-CoA desaturase assay

(76) Inventors: James M. Ntambi, Madison, WI (US); Makoto Miyazaki, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/448,198

(22) Filed: Jun. 7, 2006

Related U.S. Application Data
(60) Provisional application No. 60/688,565, filed on Jun. 8, 2005.

(51) Int. Cl. C12Q 1/00; C12Q 1/68; C12Q 1/48; C12P 7/64; A61K 31/202; C12N 1/18

(52) U.S. Cl. 435/4; 435/6; 435/134; 514/560; 435/15; 435/254.2; 436/71

(57) ABSTRACT

A yeast-based method for identifying an agent that can modulate the activity of a human or mouse stearoyl-CoA desaturase (SCD) is disclosed. Further disclosed are substrate specificities of various human and mouse SCDs, which facilitate the identification of modulators for human and mouse SCDs.

### CROSS-REFERENCE TO RELATED APPLICATIONS

This application claims the benefit of U.S. provisional application 60/688,565, filed on June 8, 2005, which is incorporated by reference in its entirety.

### STATEMENT REGARDING FEDERALLY SPONSORED RESEARCH OR DEVELOPMENT

This invention was made with United States government support awarded by the following agency: NIH DK062388. The United States has certain rights in this invention.

### BACKGROUND OF THE INVENTION

Δ9-desaturase is a fatty acid modifying enzyme that desaturates saturated acyl-CoA, a crucial step in the biosynthesis of lipids including monounsaturated fatty acids, triglycerides, cholesteryl esters, phospholipids, and wax esters. Since this enzyme commonly introduces a cis double bond at the 9, 10 position of stearoyl-CoA to form oleoyl-CoA, it has been known as stearoyl-CoA desaturase (SCD) (Miyazaki, M and Ntambi, J M (2003) Prostaglandins Leukot Essent Fatty Acids 68, 113-121; Ntambi, J M and Miyazaki, M (2003) Curr Opin Lipidol 14, 255-261; Ntambi, J M and Miyazaki, M (2004) Prog Lipid Res 43, 91-104; and Ntambi, J. M (1995) Prog Lipid Res 34, 139-150). This oxidative reaction of SCD requires cytochrome b₅, NAD(P)H-cytochrome b₅ reductase, and molecular oxygen (Ntambi, J M (1995) Prog Lipid Res 34, 139-150; Shanklin, J et al. (1994) Biochemistry 33, 12787-12794; Fox, B G et al. (1993) Proc Natl Acad Sci U S A 90, 2486-2490; and Ntambi, J M (1999) J Lipid Res 40, 1549-1558).

There are 4 isoforms of SCD in mouse (mSCD1-4) (Kaestner, K H et al. (1989) J Biol Chem 264, 14755-14761; Miyazaki, M et al. (2003) J Biol Chem 278, 33904-33911; Ntambi, J M et al. (1988) J Biol Chem 263, 17291-17300; and Zheng, Y et al. (2001) Genomics 71, 182-191, all of which are herein incorporated by reference in their entirety) and two in human (hSCD1 and hSCD5) (Zhang, L et al. (1999) Biochem J 340, 255-264; and Beiraghi, S et al. (2003) Gene 309, 11-21, both of which are herein incorporated by reference in their entirety). All mouse SCD genes are co-localized to chromosome 19 (Miyazaki, M et al. (2003) J Biol Chem 278, 33904-33911) whereas human SCD1 and SCD5 are located on chromosomes 10 and 4, respectively (Zhang, L et al. (1999) Biochem J 340, 255-264; and Beiraghi, S et al. (2003) Gene 309, 11-21). Mouse SCD1 is expressed in lipogenic tissues including liver and adipose tissues. Mouse SCD2 is mainly expressed in the brain. Mouse SCD3 expression is restricted to sebocytes in the skin and preputial and Harderian glands whereas mouse SCD4 is predominantly expressed in the heart (Ntambi, J M and Miyazaki, M (2003) Curr Opin Lipidol 14, 255-261). Human SCD1 is ubiquitously expressed in most tissues whereas hSCD5 is highly expressed in human brain and pancreas (Zhang, L et al. (1999) Biochem J 340, 255-264; Beiraghi, S et al. (2003) Gene 309, 11-21; and Zhang, S et al. (Dec. 20, 2004) Biochem J).

The difference in tissue-specific expression among SCD isoforms indicates that each isoform may have a unique role in regulating lipid metabolism. It is of great interest in the art to identify SCD modulators, especially isoform-specific modulators, to study the function of individual isoforms. SCD modulators are also of interest to the pharmaceutical industry as potential therapeutic agents. For example, SCD1 has been identified as an anti-obesity target and SCD1 inhibitors, especially those that do not cross react with other isoforms and thus less likely to have side effects, are of interest to the pharmaceutical industry as potential anti-obesity drugs.

### BRIEF SUMMARY OF THE INVENTION

A yeast-based method for identifying an agent that can modulate the activity of a human or mouse SCD is disclosed. Further disclosed are substrate specificities of various human and mouse SCDs, which facilitate the identification of modulators for human and mouse SCDs.

### BRIEF DESCRIPTION OF THE SEVERAL VIEWS OF THE DRAWINGS

FIG. 1 shows the growth of yeast ole1 mutant strain L8-14C containing mammalian Δ9-desaturases. (A) L8-14C plated onto media lacking unsaturated fatty acids. (B) Western blot analysis of yeast L8-14C containing mammalian Δ9-desaturases.

FIG. 2 shows conversion of saturated fatty acid to Δ9 monounsaturated fatty acids. Fatty acids (0.2 mM) were added in presence of 1% tergitol NP-40 and yeast cells were cultured for 2 days. Values represent the mean conversion (%)±SE (n=5).

FIG. 3 shows Δ9-desaturase activity in Hela cells over-expressing mammalian Δ9-desaturases. Microsomal fractions (100 μg) were incubated with either [¹⁴C]stearoyl-CoA or [¹⁴C]pamitoyl-CoA in the presence of NADH. Each value represents the mean±SE (n=4).

FIGS. 4A and 4B show sequence alignment of Δ9-desaturases. The underline shows the putative transmembrane domains. Black boxes indicate the conserved histidine boxes. FAT5 and Le-FAD1 are palmitoyl-CoA-specific Δ9-desaturase from C. elegance and stearoyl-CoA-specific Δ9-desaturase from Basidiomycte Lentinula edodes, respectively.

### DETAILED DESCRIPTION OF THE INVENTION

The present invention provides a yeast-based method for identifying an agent that can modulate the activity of a human or mouse SCD. This method uses yeast growth or survival as the end point of measurement and can therefore be easily adapted for high throughput screens. The inventors have tested the method with a known hSCD1 inhibitor and confirmed that the inhibitor would have been successfully identified by the method of the present invention.

The method of the present invention is also well suited for studying substrate specificity of different human and mouse SCD isoforms, which has not been well characterized in the art. As shown in the example below, the inventors have found that human and mouse SCD1 and mouse SCD2 have a broader range of fatty acyl-CoA substrates ranging from C13 to C19 (tridecanoyl-CoA, myristoyl-CoA, pentadecanoyl-CoA, palmitoyl-CoA, margaroyl-CoA, stearoyl-CoA, and nonadecanoyl-CoA). Mouse SCD3 catalyzes desaturation of fatty acyl-CoA substrates of C12 to C16. Although it may be appropriate to rename mSCD3 to palmitoyl-CoA disaturase given that it utilizes palmitoyl-CoA but not stearoyl-CoA, the specification and claims continue to refer to this enzyme by the art-recognized name—SCD3—in keeping with the existing literature. Mouse SCD4 catalyzes desaturation of fatty acyl-CoA substrates of C14 to C19 (the efficiency is low for C14 fatty acyl-CoA and C19 fatty acyl-CoA). Human SCD5 uses C14 to C19 fatty acyl-CoA as substrates (the efficiency is low for C14 fatty acyl-CoA). Although the use of C15 fatty acyl-CoA has not been tested, it is expected that mSCD1-4, hSCD1, and hSCD5 can all use C15 fatty acyl-CoA as a substrate based on the C14 and C16 data.

In one aspect, the present invention relates to a method for identifying an agent that can modulate (inhibit or enhance) the activity of a human or mouse SCD. The method includes the steps of:

a) providing yeast cells that have been genetically engineered to express a human or mouse SCD protein wherein the endogenous yeast SCD nucleic acid sequence has been disrupted;

b) culturing the yeast cells in a medium that contains a saturated fatty acyl-CoA substrate of the human or mouse SCD or a corresponding saturated fatty acid that can be converted to said acyl-CoA substrate in the yeast cells;

c) exposing the yeast cells to a test agent; and

d) determining the effect of the test agent on yeast cell growth, survival, or both wherein a negative effect on yeast cell survival, growth, or both indicates that the test agent can inhibit the activity of the human or mouse SCD and a positive effect on yeast cell survival, growth, or both indicates that the test agent can enhance the activity of the human or mouse SCD.

The yeast cells employed in the method of the present invention contains a disruption in the yeast SCD gene (ole1) or nucleic acid sequence, resulting in a reduced or no detectable expression of the functional yeast SCD protein. “Reduced” indicates 30% or less, 20% or less, 10% or less, 5% or less, 3% or less, or 1% or less of the level of functional yeast SCD protein expression in control yeast cells, i.e., yeast cells in which the yeast SCD gene has not been disrupted. In a preferred embodiment, the yeast cells are from an ole1 knock-out strain that has no detectable level of ole1 expression, such as the strain described in Stukey, J E et al. (1990) J Biol Chem 265, 20144-20149, which is herein incorporated by reference in its entirety.

The yeast SCD gene may be disrupted using a variety of technologies familiar to those skilled in the art. For example, a stop codon may be introduced into the gene by homologous recombination. Alternatively, a deletion may be introduced into the gene by homologous recombination. In some embodiments, the gene may be disrupted by inserting a gene encoding a marker protein, for example, therein via homologous recombination. A skilled artisan is familiar with how a yeast cell with disrupted yeast SCD gene.

A nucleic acid encoding the human or mouse SCD protein can be integrated into a yeast chromosome or provided on an episomal vector. In either case, the expression of the human or mouse SCD protein is controlled by a yeast promoter. In one embodiment, the yeast promoter is a promoter other than the ole1 promoter. In a preferred embodiment, the yeast promoter is the yeast glyceraldehydes-3-phosphate dehydrogenase promoter. In another preferred embodiment, the nucleic acid encoding the human or mouse SCD protein is cloned into the yeast expression vector 426GPD of American Tissue Culture Collection in which the yeast glyceraldehydes-3-phosphate dehydrogenase promoter is provided to drive the expression of an inserted gene.

Although a DNA sequence encoding a tag peptide may be attached to and expressed with the nucleic acid encoding the human or mouse SCD protein to facilitate the identification of the expressed protein or to serve other purposes, no DNA sequence that encodes a peptide unique to the yeast SCD protein is attached to the nucleic acid so that the expressed human or mouse SCD protein is not fused with any peptide unique to the yeast SCD protein. For example, the expressed human or mouse SCD protein is not fused with amino acids 1-10, 1-15, 1-20, 1-25, or 1-27 of the N-terminus of the yeast SCD protein. Preferably, the expressed human or mouse protein is not fused with any 3, 5, 10, 15, 20, 25, or 27 consecutive amino acids of the yeast SCD protein. In one embodiment, the expressed human or mouse SCD protein does not contain any extra amino acid.

The yeast culture medium used in the method of the present invention can contain one or more monounsaturated fatty acids and their corresponding acyl-CoAs that support the growth of the yeast cells as long as the level of the monounsaturated fatty acids and their corresponding acyl-CoAs is lower than that needed for maximal cell growth. In a preferred embodiment, the culture medium contains no monounsaturated fatty acids and their corresponding acyl-CoAs or at a very low level not enough to support survival of the yeast cells.

The SCD substrate specificity disclosed here allows the method of the present invention to be practiced with specific substrates. For example, for hSCD1, mSCD1, and mSCD2, any one of C13-C19 saturated fatty acyl-CoAs or a combination thereof can be used as a substrate. For mSCD3, any one of C12-C16 (C13-C16 are preferred) saturated fatty acyl-CoAs or a combination thereof can be used as a substrate. For mSCD4, any one of C14-C19 saturated fatty acyl-CoAs or a combination thereof can be used as a substrate. In a preferred embodiment, one or more of C14-C18, C15-C18, or C16-C18 saturated fatty acyl-CoAs are used as substrates. For hSCD5, any one of C14-C19 saturated fatty acyl-CoAs or a combination of any of the foregoing can be used as a substrate. In one embodiment, a C13, C14, C15, C17, or C19 saturated fatty acyl-CoA or a combination of any of the foregoing is used as a substrate for hSCD1, mSCD1, and mSCD2; a C12, C13, C14, or C15 (C13-C15 are preferred) saturated fatty acyl-CoA or a combination of any of the foregoing is used as a substrate for mSCD3; a C14, C15, C17, or C19 (C15 and C17 are preferred) saturated fatty acyl-CoA or a combination of any of the forgoing is used as a substrate for mSCD4; and a C14, C15, C17, or C19 (C15, C17, and C19 are preferred) saturated fatty acyl-CoA or a combination of any of the foregoing is used as a substrate for hSCD5.

It is well within the capability of a skilled artisan to set up various controls to determine whether an agent has a negative (inhibitory) or positive (enhancing) effect on the SCD activity being tested. For example, the survival or growth rate of the same yeast culture before and after exposure to a test agent can be compared. Alternatively, the survival or growth rate of the test agent-treated group can be compared to that of a control group run in parallel but not treated with the test agent.

In one embodiment, the method is used to identify a modulator of a human or mouse SCD with the proviso that the human or mouse SCD is not hSCD1 and mSCD1. In another embodiment, the method is used to identify a modulator of hSCD1, hSCD5, mSCD 1, mSCD2, mSCD3, or mSCD4. In still another embodiment, the method is used to identify a modulator of hSCD5, mSCD2, mSCD3, or mSCD4. The nucleic acid sequences of hSCD1, hSCD5, mSCD1, mSCD2, mSCD3, and mSCD4 are available in the art and can be found in the GenBank with accession numbers AF097514, AF389338, M21280, M26269, AF272037, and AY430080, respectively.

The method of the present invention can be used to test the effect of a test agent on at least two, three, four, five, or six individual human and mouse SCDs as described above. This allows the determination of whether a SCD modulator is isoform specific.

The method of the present invention can further include a step, after an SCD modulator has been identified by the yeast system, of verifying the SCD modulating effect in a mammalian system with which a skilled artisan is familiar. For example, mammalian cells (e.g., Hela cells or HEK-293 cells), especially those with low endogenous SCD activity (e.g., Hela cells), can be transfected with an expression vector containing the human or mouse SCD gene of interest and then cultured under the conditions that allow the expression of the human or mouse SCD gene. The microsomes of these cells can then be isolated and the SCD activity be measured by a microsomal assay in the presence and absence of a test agent. An example of such a mammalian cell-based assay is described in the example below. Another example is described in Miyazaki M et al. (2003) J Biol Chem 278, 33904-33911, which is herein incorporated by reference in its entirety. Other mammalian systems such as those described in the context of SCD1 in WO2004/010927, which is herein incorporated by reference in its entirety, can also be used.

Some of the substrates identified for the human and mouse SCD isoforms based on the substrate specificity study disclosed herein have not been recognized as substrates for these SCDs in the prior art. These newly identified substrates include tridecanoyl-CoA, myristoyl-CoA, pentadecanoyl-CoA, margaroyl-CoA, and nonadecanoyl-CoA for hSCD1, mSCD1, and mSCD2; myristoyl-CoA, pentadecanoyl-CoA, margaroyl-CoA, and nonadecanoyl-CoA for hSCD5; lauroyl-CoA, tridecanoyl-CoA, myristoyl-CoA, and pentadecanoyl-CoA for mSCD3; and myristoyl-CoA, pentadecanoyl-CoA, margaroyl-CoA, and nonadecanoyl-CoA for mSCD4. Such information enables a method of converting the above saturated fatty acyl-CoA substrates to the corresponding monounsaturated fatty acyl-CoAs by exposing a composition that consists essentially one or more of the above saturated fatty acyl-CoAs to an appropriate SCD under the conditions that allow the formation of the monounsaturated fatty acyl-CoAs. Various such conditions are known in the art and other suitable conditions can also be readily recognized or determined by a skilled artisan. Examples of suitable conditions can be found in Miyazaki, M et al. (2001) J Biol Chem 276, 39455-39461 (incorporated by reference in its entirety); Miyazaki, M et al. (2003) J Biol Chem 278, 33904-33911; and WO2004/010927. The formation of the above monounsaturated fatty acyl-CoAs can be observed by any known technique in the art.

The substrate specificity information provided here also enables new methods for identifying an agent that can modulate the activity of hSCD1, hSCD5, mSCD1, mSCD2, mSCD3, or mSCD4. Such methods involve providing a preparation that contains the activity of one of the human and mouse SCDs and a composition that consists essentially of one or more newly-identified saturated fatty acyl-CoA substrates or the corresponding saturated fatty acids that can be converted to the acyl-CoA substrates under the conditions that allow the formation of the corresponding monounsaturated fatty acyl-CoAs. The preparation is then exposed to a test agent and the SCD activity is measured and compared to that of a control preparation that is not exposed to the test agent wherein a difference between the SCD activity of the test agent-treated group and that of the control group indicates that the agent can modulate the activity of the SCD. In one embodiment, the SCD activity is measured by observing the formation of one or more monounsaturated fatty acyl-CoAs.

Various preparations that contain the activity of hSCD1, hSCD5, mSCD1, mSCD2, mSCD3, or mSCD4 are well known in the art and additional preparations can also be developed by a skilled artisan. Examples of such preparations are described in Miyazaki, M et al. (2001) J Biol Chem 276, 39455-39461; Miyazaki, M et al. (2003) J Biol Chem 278, 33904-33911; and WO2004/010927. Depending on the particular preparation employed, either the saturated fatty acyl-CoA substrates or the corresponding saturated fatty acids can be incubated with the preparation to support the SCD activity. For example, microsomal assays generally employ the direct substrates—saturated fatty acyl-CoAs—and the yeast assay disclosed here can use the corresponding saturated fatty acids.

The invention will be more fully understood upon consideration of the following non-limiting example.

### EXAMPLE

### Using Yeast and Mammalian Cell SCD Assays to Determine SCD Substrate Specificity

Materials and Methods

Cloning of the full-length human and mouse SCD cDNAs: The full coding region of human SCDs (hSCD1 and hSCD5 with GenBank Accession Nos. AF097514 and AF389338, respectively) and mouse SCDs (mSCD1, mSCD2, mSCD3, and mSCD4 with GenBank Accession Nos. M21280, M26269, AF272037, and AY430080, respectively) were generated by PCR using human and mouse tissue cDNAs as templates and with 5′ primers which contain a sequence of N-terminal hemagglutinin epitope (HA) tag and either EcoRI or SalI restriction enzyme site and 3′-primers which contain a stop codon and a XhoI restriction enzyme site. The resulting PCR product was cloned into either a yeast expression vector, p426GPD (American Type Culture Collection, and Mumberg D et al. (1995) Gene 156:119-122, which is herein incorporated by reference in its entirety) or a mammalian expression vector, pcDNA3 (Invitorgen). The integrity of the PCR product was confirmed by DNA sequencing.

Functional Analysis: The p426GPD constructs harboring either human or mouse SCDs were transformed into Saccharomyces cerevisiae strain L8-14C (provided by Professor Charles Martin, Division of Life Sciences, Department of Cell Biology and Neuroscience, Rutgers University, Nelson Laboratories), which contains a disruption of the yeast Δ9-desaturase gene ole1 and requires unsaturated fatty acids for growth (Stukey, J. E. et al. (1990) J Biol Chem 265, 20144-20149), by using a Lithium acetate standard methods (Elble, R. (1992) Biotechniques 13, 18-20). The transformed yeast cells were plated onto a synthetic dextrose medium containing 1% tergitol NP-40, 0.5 mM oleic acid, and 0.5 mM palmitoleic acid but lacking uracil. To test the genetic complementation of the mutant yeast strain, transformed yeast cells were plated onto YPD (Yeast Extract/Peptone/Dextrose) medium lacking unsaturated fatty acids. Plates were incubated at 30° C. for 3 days.

HeLa cells were cultured at 37° C. in a humidified 5% CO₂ atmosphere in Dulbecco's modified Eagle's medium supplemented with 10% (v/v) fetal bovine serum and penicillin/streptomycin. The cells were resuspended in cytomix buffer (120 mM KCl, 0.15 mM CaCl₂, 25 mM Hepes/KOH, pH 7.6, 2 mM EGTA, 5 mM MgCl₂), and 400 μl of suspension was transferred to a 0.4-cm electroporation cuvette (Invitrogen). 35 μg of pcDNA3 DNA harboring either human or mouse SCD constructs were transfected into Hela cells which have very low Δ9-desaturase activity. DNA was added to the cell suspension in the cuvette and mixed well. The mixture was then exposed to a single electric pulse of 300 V with a capacitance of 1000 microfarads using an Invitrogen pulse system. The cells were allowed to recover in culture medium at 37° C. (5% CO₂ atmosphere) for 48 h before harvesting and performing SCD activity assays.

Fatty acid analysis: Yeast cells were grown in liquid YPD medium lacking unsaturated fatty acids for 3 days. Cells were pelleted and washed twice with water, followed by suspension in 0.5 ml of 2M NaOH in methanol. The mixture was then heated to 80° C. for 1 h and acidified with formic acid. Fatty acids were extracted according to Bligh & Dyer's method and trans-methylated with 1 ml of 14% BF3 in methanol (Sigma). The resulting fatty acid methyl esters were extracted with hexane and analyzed by gas-liquid chromatography (GLC) (Miyazaki, M. et al. (2001) J Biol Chem 276, 39455-39461; and Elble, R. (1992) Biotechniques 13, 18-20). The double bond positions of the monounsaturated fatty acid pyrrolidide derivatives were analyzed by tandem GC-MS as described in Miyazaki, M. et al. (2002) J Lipid Res 43, 2146-2154, incorporated by reference in its entirety. Exogenous saturated fatty acids (0.2 mM) were added into YPD in presence of 1% Tergitol NP-40 (Sigma).

Δ9-desaturase activity: Microsomes were purified from Hela cells by differential centrifugation and resuspended in a 0.1 M potassium phosphate buffer (pH 6.8). Δ9-desaturase activity was assayed at 25° C. for 7 minutes with either [¹⁴C] stearoyl-CoA or [¹⁴C] palmito-CoA, 2 mM NADH, and 100 μg microsomal protein (Miyazaki, M. et al. (2001) J Biol Chem 276, 39455-39461).

Immnoblot analysis: Yeast protein extract was electrophoresed on 8% SDS-PAGE and transferred to a nitrocellulose membrane (Millipore). The membrane was blocked at room temperature for 1 h in TBST containing 1% BSA and then incubated with 100 ng/ml anti-HA monoclonal antibody (clone 3F10, Roche) in TBS containing 1% BSA for 1 h at room temperature. After washing with TBS containing 0.1% Tween 20, the membrane was incubated with 1:20000 dilution of horseradish peroxidase-conjugated anti-rat IgG (Sigma) for 30 min at room temperature. The signal was visualized with ECL Western blot detection kit (Pierce).

Statistical analysis: All data are expressed as means±SE. An unpaired student's t-test was used to determine significance.

Results

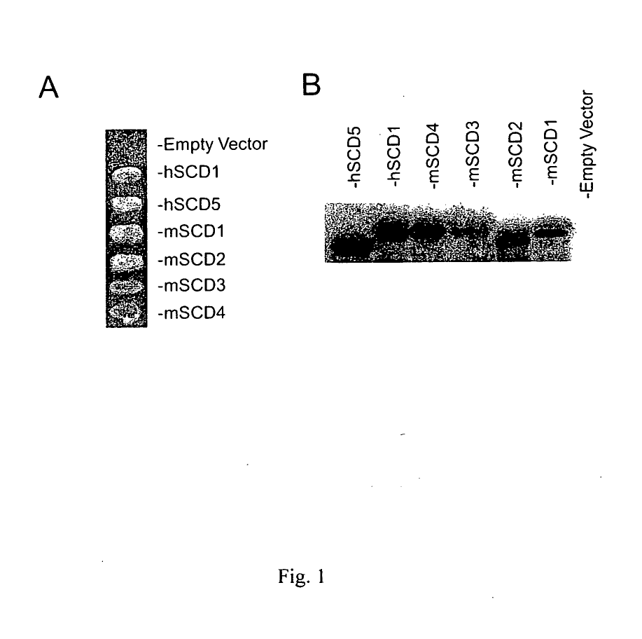

To study the function of the human SCD (hSCD1 and hSCD5) and mouse SCD (mSCD1, mSCD2, mSCD3, and mSCD4) genes, the open reading frames (ORFs) of the genes were subcloned in the episomal yeast expression vector 426GPD which encodes uracil prototrophy under the constitutive glyceraldehydes-3-phosphate dehydrogenase promoter and the resulting plasmid was used to transform L8-14C, a Δ9-desaturase (OLE1)-deficient yeast strain. As shown in FIG. 1, yeast transformed with all plasmids containing SCDs were able to grow on YPD plate lacking unsaturated fatty acids, indicating that the mouse and human Δ9-desaturases were functional in yeast by their ability to complement ole1 mutation. The fatty acid compositions of transformants as determined by GLC are shown in Table 1. Yeast expressing mSCD1 and mSCD2 had similar fatty acid compositions. Mouse SCD1 and mSCD2 converted 85% and 77%, respectively, of 18:0 to 18:1Δ9 and 51% and 36%, respectively, of 16:0 to 16:1Δ9. Mouse SCD4 was able to use 8% of 16:0 and 13% of 18:0 but the conversion rates were lower than those of mSCD1 and mSCD2. Accordingly, mouse SCD1, SCD2, and SCD4 enzymes were able to convert 16:0 and 18:0 to 16:1Δ9 and 18:1Δ9, respectively, although the three enzymes preferably utilized 18:0 compared to 16:0. Mouse SCD3 converted 49% 16:0 to 16:1Δ9, but the conversion of 18:0 to 18:1Δ9 was less than 2%, suggesting that this isoform prefers C16:0 as a substrate. Similar to mSCD1, hSCD1 was able to generate both 18:1Δ9 and 16:1Δ9 but the conversion towards 18:0 was 1.7-fold higher. Yeast expressing hSCD5 showed unique substrate specificity. The conversion of 18:0 to 18:1Δ9 was more than 4-hold higher than that of 16:0. The immunoblot analysis with an HA antibody showed that all Δ9-desaturase proteins were expressed in yeast at the expected size (FIG. 1B). The Δ9 position of the double bond in all monounsaturated fatty acids were determined by GC/MS analysis of the dimethyl disulfide derivatization. As expected, the mass spectra of 16:1 and 18:1 in yeast expressing any SCD showed the characteristic Δ9 unsaturated fragments ions at m/z 217 and 185 (data not shown).

TABLE 1
Fatty acid composition of total lipids in L8-14C transformants

| | Fatty acid composition (%) | | | | | % conversion | % conversion |
|---|---|---|---|---|---|---|---|
| | 16:0 | 16:1n − 7 | 18:0 | 18:1n − 9 | 18:1n − 7 | of 16:0 | of 18:0 |
| mSCD1 | 49.9 | 27.9 | 3.3 | 18.3 | 0.6 | 36.4 | 84.8 |
| mSCD2 | 41.6 | 42.9 | 3.3 | 11.2 | 1.0 | 51.4 | 77.3 |
| mSCD3 | 42.7 | 40.0 | 16.2 | 0.0 | 1.1 | 49.0 | 0.3 |
| mSCD4 | 60.1 | 5.9 | 27.8 | 5.6 | 0.6 | 9.7 | 16.8 |
| hSCD1 | 50.4 | 38.0 | 2.6 | 7.8 | 1.2 | 43.8 | 75.0 |
| hSCD5 | 61.0 | 14.8 | 3.2 | 20.6 | 0.4 | 19.9 | 86.6 |

Data represents the content of fatty acid in % of total fatty acids.
Standard errors of the mean were all less than 10% and are omitted for clarity.

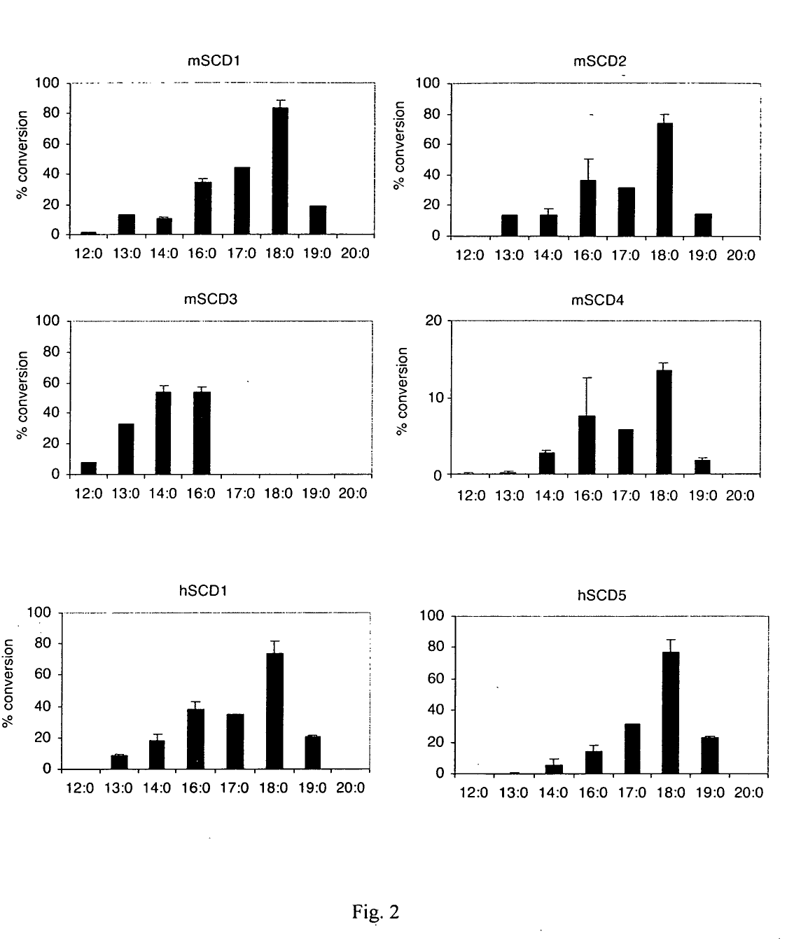

To determine whether the mouse and human Δ9-desaturases are able to desaturate other saturated fatty acids including lauric acid (12:0), tridecanoic acid (13:0), myristic acid (14:0), heptadecanoic acid (17:0), nonadecanoic acid (19:0), and eicosanoic acid (20:0), we exogenously provided saturated fatty acids (0.2 mM) to yeast expressing each SCD isoform (FIG. 2). Exogenous 16:0 and 18:0 were converted to 16:1Δ9 and 18:1Δ9, respectively, at similar ratio as the endogenous ones. Mouse SCD1, mSCD2, and hSCD1 converted 13%, 14%, and 9% of 13:0 to 13:1Δ9, respectively, 11%, 14%, and 18% of 14:0 to 14:1Δ9, respectively, and 43%, 31%, and 35% of C17:0 to 17:1Δ9, respectively. Mouse SCD3 converted 14% of 12:0, 32% of 13:0, and more than 50% of 14:0 to 12:1Δ9, 13:1Δ9, and 14:1Δ9, respectively. The conversion of 17:0 to 17:0Δ9 was undetectable in the yeast expressing mSCD3. Mouse SCD4 utilized only 2.9% of 14:0 and 5.7% of 17:0, suggesting that mSCD4 may use other acyl-CoA as a major substrates. Human SCD5 converted less than 5% of 14:0 to 14:1. Human SCD5 did not desaturate C12:0 or C13:0 but converted 31% of 17:0 to 17:1Δ9. None of Δ9-desaturase was able to utilize a C20:0 saturated fatty acid.

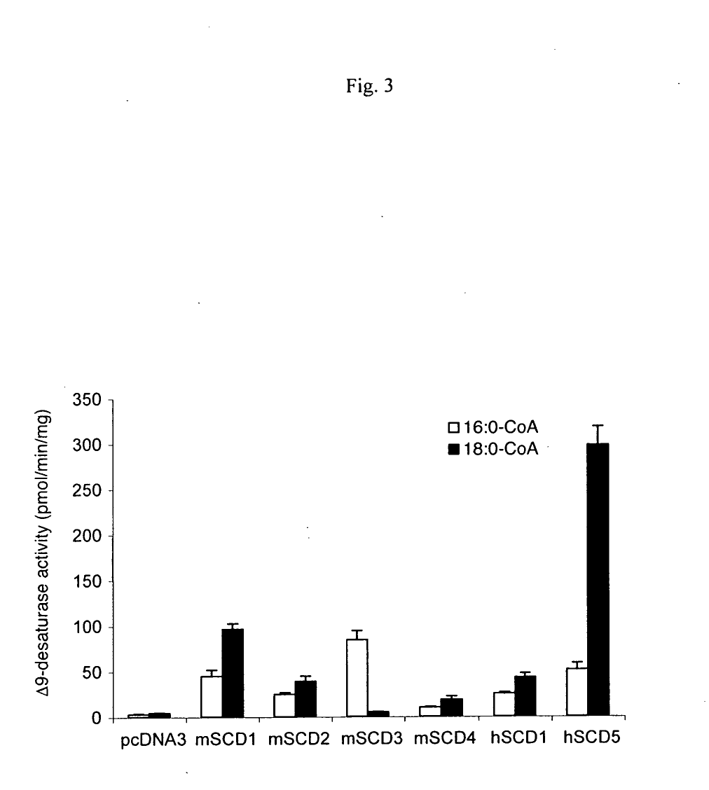

To determine whether the mouse and human Δ9-desaturase displayed similar substrate specificities in mammalian cells, we re-cloned the Δ9-desaturases into a mammalian expression vector and transfected them into Hela cells (human cervical cancer cells) which have very low Δ9-desaturase activity compared to other human cell lines including HEK-293, HepG2, CHO, MDA, and MCF-7 cells (data not shown). The substrate preferences of all the Δ9-desaturases in mammalian cells are consistent with that found in the yeast experiments. Hela cells overexpressing mSCD1, mSCD2, mSCD4, and hSCD1 utilized 16:0-CoA and 18:0-CoA but with a 2.2-, 1.6-, 2.0-, and 2.2-fold higher Δ9-desaturase activity towards 18:0-CoA than 16:0-CoA. Mouse SCD3 showed a 16-fold higher activity towards 16:0-CoA than 18:0-CoA while a 6-fold higher utilization of 18:0-CoA was observed in Hela cells expressing hSCD5 (FIG. 3).

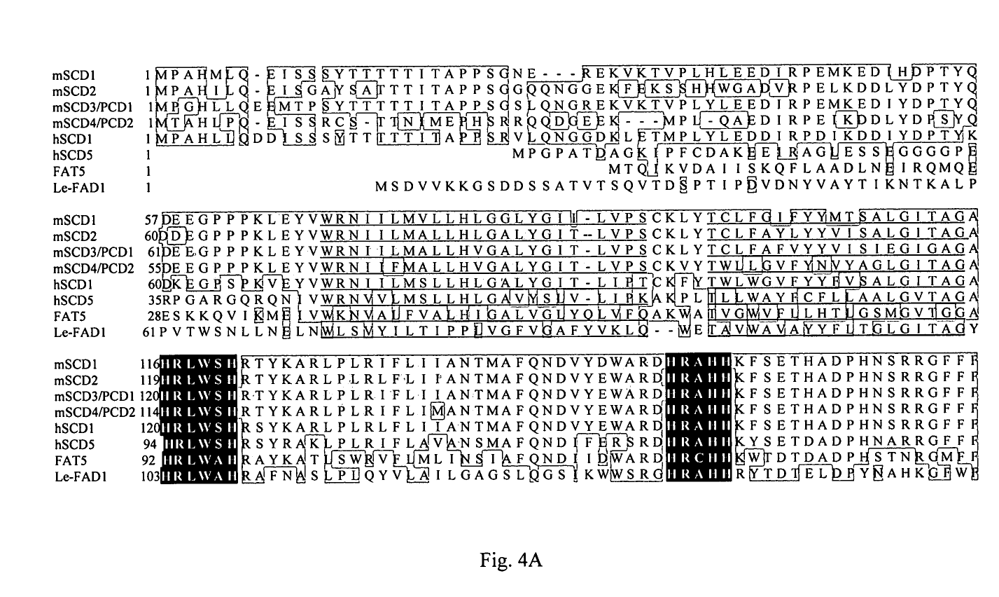

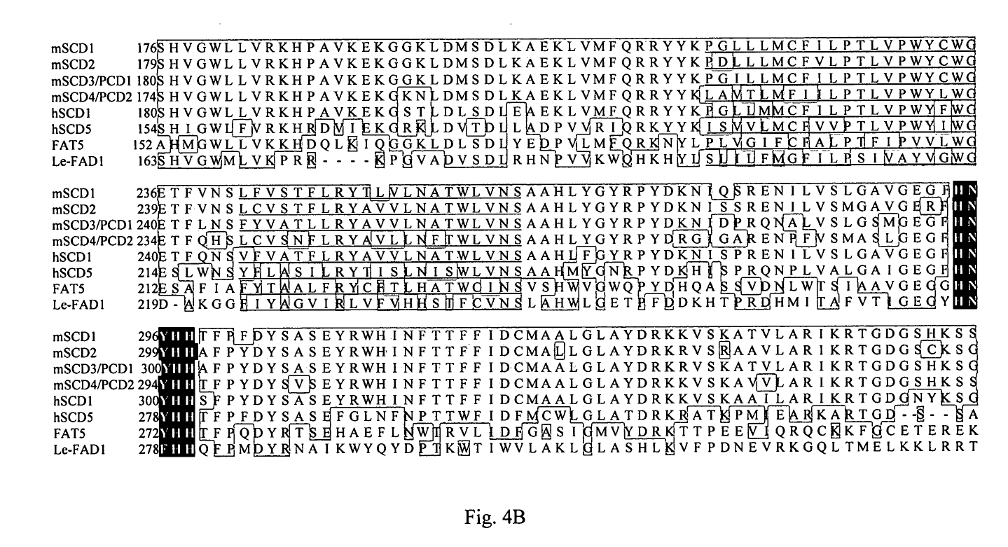

FIGS. 4A and 4B show the comparison of human and mouse Δ9-desaturases to determine which portion and/or amino acid residue of the protein is responsible for the substrate specificity. FAT-5 palmitoyl-CoA-specific Δ9-desaturase from C. elegance (Watts, J. L., and Browse, J. (2000) Biochem Biophys Res Commun 272, 263-269) and Le-FAD1 oleoyl-CoA-specific from the fungus Basidiomycte Lentinula edodes (Sakai, H., and Kajiwara, S. (2003) Biosci Biotechnol Biochem 67, 2431-2437) are aligned with human and mouse Δ9-desaturases. The amino acid sequence of FAT5 has less than 50% identity to mSCD3 while Le-FAD1 has less than 40% amino acid identity to hSCD5. Three catalytically essential histidine boxes and 4 transmembrane domains are conserved in all of Δ9-desaturases. Despite distinct substrate specificities, the presumed catalytic sites of mouse Δ9-desaturases, particularly mSCD1 and mSCD3, are very similar (>94%). The amino acid alterations between these two proteins are concentrated in 20 amino acid residues prior to the third histidine box, suggesting an amino acid in this portion of the protein may distinguish substrates due to their chain-length.

The existence of palmitoyl-CoA desaturases could be due to the unique tissue-specific distribution and difference in the melting points of the monounsaturated fatty acid products. Mouse SCD3 is highly expressed in skin sebaceous glands which produce lipid secretions sebum (Zheng, Y. et al. (2001) Genomics 71, 182-191). The most abundant fatty acid in sebum (mostly wax ester) is 16:1Δ9 and the amount is more than 5 times of oleic acid (Green, S. C. et al. (1984) J Invest Dermatol 83, 114-117). Since skin is poikilothermal, sebum is easily affected by the environmental temperature. The melting point of 16:1Δ9 (m.p. 0.5° C.) is lower than that of 18:1Δ9 (m.p 16.2° C.). Thus, 16:1Δ9 appears to be more resistant to cold temperature and could be preferentially utilized by acyl-CoA wax alcohol acyltransferases (AWAT1 and AWAT2) (Turkish, A. R. et al. (2005) J Biol Chem 280, 14755-14764; and Cheng, J. B. and Russell, D. W. (2004) J Biol Chem 279, 37798-37807) in the synthesis of skin waxes. Skin of SCD1−/−mice exhibited alopecia, atrophy sebaceous gland, and decrease in sebum production (Miyazaki, M. et al. (2001) J Nutr 131, 2260-2268; and Zheng, Y. et al. (1999) Nat Genet 23, 268-270). Interestingly mSCD3 expression was lost in the skin of SCD1−/− mice (Zheng, Y. et al. (2001) Genomics 71, 182-191). These data suggested that 16:1Δ9 synthesized from mSCD3 is an important fatty acid in skin function such as wax production and hair growth. In addition, we previously found that the Harderian sebocytes of SCD1 −/− mice have a high Δ9-desaturase activity towards 16:0-CoA but not 18:0-CoA (Miyazaki, M. et al. (2001) J Biol Chem 276, 39455-39461). We therefore concluded that the residual palmitoyl-CoA desaturase activity was derived from mSCD3.

Two isoforms of Δ9-desaturase exist in the human genome (Zhang, L. et al. (1999) Biochem J 340, 255-264; and Beiraghi, S. et al. (2003) Gene 309, 11-21). The substrate preference, tissue distribution (except for brain), and protein sequence of hSCD1 were very similar to those of mSCD1 (Zhang, L. et al. (1999) Biochem J 340, 255-264) whereas hSCD5 is very distinct from hSCD1 in this regard (Zhang, S. et al. (2005) Biochem J. 388, 135-142). In addition, we found that hSCD5 has higher preference towards 18:0 over 16:0. The inversion of hSCD5 is reported to be involved in cleft lip development which is a common human birth defect affecting 1 in every 700 live births. Therefore, 18:1Δ9 may be a regulator of human lip development.

The present invention is not intended to be limited to the foregoing example, but encompasses all such modifications and variations as come within the scope of the appended claims.

We claim:

1. A method for identifying an agent that can modulate the activity of a human or mouse stearoyl-CoA disaturase (SCD), the method comprising the steps of:
a) providing yeast cells that have been genetically engineered to express a human or mouse SCD protein wherein the native SCD nucleic acid sequence of the yeast cells has been disrupted;
b) culturing the yeast cells in a medium that contains a saturated fatty acyl-CoA substrate of the human or mouse SCD or a corresponding saturated fatty acid substrate that can be converted to the saturated fatty acyl-CoA substrate in the yeast cells;
c) exposing the yeast cells to a test agent; and
d) determining the effect of the test agent on yeast cell growth, survival, or both wherein a negative effect on yeast cell survival, growth, or both indicates that the test agent can inhibit the activity of the human or mouse SCD and a positive effect on yeast cell survival, growth, or both indicates that the test agent can enhance the activity of the human or mouse SCD.

2. The method of claim 1, wherein the method is used for identifying an agent that can inhibit the activity of a human or mouse SCD.

3. The method of claim 1, wherein the human or mouse SCD is selected from human SCD1, human SCD5, mouse SCD 1, mouse SCD2, mouse SCD3, or mouse SCD4.

4. The method of claim 1, wherein the yeast cells contain a vector that comprises a nucleic acid sequence encoding the human or mouse SCD protein and a yeast promoter operably linked to the nucleic acid sequence wherein upon expression the human or mouse SCD protein consists of the full length amino acid sequence of the protein.

5. The method of claim 4, wherein the yeast promoter is the yeast glyceraldehydes-3-phosphate dehydrogenase promoter.

6. The method of claim 1, wherein the human or mouse SCD is human SCD1 and the saturated fatty acid substrate of the human or mouse SCD consists essentially of a fatty acid selected from tridecanoic acid, myristic acid, pentadecanoic acid, margaric acid, nonadecanoic acid, or a combination of any of the foregoing.

7. The method of claim 1, wherein the human or mouse SCD is human SCD5 and the saturated fatty acid substrate of the human or mouse SCD consists essentially of a fatty acid selected from myristic acid, pentadecanoic acid, margaric acid, nonadecanoic acid, or a combination thereof.

8. The method of claim 1, wherein the human or mouse SCD is mouse SCD1 and the saturated fatty acid substrate of the human or mouse SCD consists essentially of a fatty acid selected from tridecanoic acid, myristic acid, pentadecanoic acid, margaric acid, nonadecanoic acid, or a combination of any of the foregoing.

9. The method of claim 1, wherein the human or mouse SCD is mouse SCD2 and the saturated fatty acid substrate of the human or mouse SCD consists essentially of a fatty acid selected from tridecanoic acid, myristic acid, pentadecanoic acid, margaric acid, nonadecanoic acid, or a combination of any of the foregoing.

10. The method of claim 1, wherein the human or mouse SCD is mouse SCD3 and the saturated fatty acid substrate of the human or mouse SCD consists essentially of a fatty acid selected from lauric acid, tridecanoic acid, myristic acid, pentadecanoic acid, or a combination thereof.

11. The method of claim 1, wherein the human or mouse SCD is mouse SCD4 and the saturated fatty acid substrate of the human or mouse SCD consists essentially of a fatty acid selected from myristic acid, pentadecanoic acid, margaric acid, nonadecanoic acid, or a combination of any of the foregoing.

12. The method of claim 1, further comprising the step of repeating steps a) to d) for at least one other human or mouse SCD.

13. The method of claim 1, wherein the method further comprising the step of testing whether an identified SCD modulator can modulate the activity of the human or mouse SCD in a mammalian cell-based assay system.

14. A method for converting a saturated fatty acyl-CoA to a corresponding monounsaturated fatty acyl-CoA, the method comprising the step of:
exposing a human or mouse stearoyl-CoA disaturase (SCD) selected from the group consisting of human SCD1, human SCD5, mouse SCD1, mouse SCD2, mouse SCD3, and mouse SCD4 to a fatty acyl-CoA composition under the conditions which allow a saturated fatty acyl-CoA to be converted to a corresponding monounsaturated fatty acyl-CoA, wherein
(a) when the SCD is human SCD1, mouse SCD1, or mouse SCD2, the fatty acyl-CoA composition consists essentially of tridecanoyl-CoA, myristoyl-CoA, pentadecanoyl-CoA, margaroyl-CoA, nonadecanoyl-CoA, or a combination of any of the foregoing;
(b) when the SCD is human SCD5, the fatty acyl-CoA composition consists essentially of myristoyl-CoA, pentadecanoyl-CoA, margaroyl-CoA, nonadecanoyl-CoA, or a combination of any of the foregoing;
(c) when the SCD is mouse SCD3, the fatty acyl-CoA composition consists essentially of lauroyl-CoA, tridecanoyl-CoA, myristoyl-CoA, pentadecanoyl-CoA, or a combination of any of the foregoing; and
(d) when the SCD is mouse SCD4, the fatty acyl-CoA composition consists essentially of myristoyl-CoA, pentadecanoyl-CoA, margaroyl-CoA, nonadecanoyl-CoA, or a combination of any of the foregoing.

15. The method of claim 14, wherein the method further comprising the step of observing the formation of the corresponding monounsaturated fatty acyl-CoA.

16. A method for identifying an agent that can modulate the activity of a human or mouse stearoyl-CoA disaturase (SCD), the method comprising the steps of:
providing a preparation that contains human or mouse SCD activity and a substrate selected from a saturated fatty acyl-CoA or a saturated fatty acid under the conditions which allow the formation of the corresponding monounsaturated fatty acyl-CoA, wherein the human or mouse SCD is selected from the group consisting of human SCD1, human SCD5, mouse SCD1, mouse SCD2, mouse SCD3, and mouse SCD4;
exposing the preparation to a test agent; and
measuring the SCD activity and comparing the SCD activity to that of a control preparation that is not exposed to the test agent, wherein
(a) when the SCD is human SCD1, mouse SCD1 or mouse SCD2, the substrate consists essentially of tridecanoic acid, tridecanoyl-CoA, myristic acid, myristoyl-CoA, pentadeanoic acid, pentadecanoyl-CoA, margaric acid, margaroyl-CoA, nonadecanoic acid, nonadecanoyl-CoA, or a combination of any of the foregoing;
(b) when the SCD is human SCD5, the substrate consists essentially of myristic acid, myristoyl-CoA, pentadecanoic acid, pentadecanoyl-CoA, margaric acid, margaroyl-CoA, nonadecanoic acid, nonadecanoyl-CoA, or a combination of any of the foregoing;
(c) when the SCD is mouse SCD3, the substrate consists essentially of lauric acid, lauroyl-CoA, tridecanoic acid, tridecanoyl-CoA, myristic acid, myristoyl-CoA, pentadecanoic acid, pentadecanoyl-CoA, or a combination of any of the foregoing; and
(d) when the SCD is mouse SCD4, the substrate consists essentially of myristic acid, myristoyl-CoA, pentadecanoic acid, pentadecanoyl-CoA, margaric acid, margaroyl-CoA, nonadecanoic acid, nonadecanoyl-CoA, or a combination of any of the foregoing.

17. The method of claim 16, wherein the SCD activity is measured by observing the formation of the corresponding monounsaturated fatty acyl-CoA.


SEQUENCE LISTING (8 sequences)

SEQ ID NO: 1 - protein, 355 aa, Mus musculus
MPAHMLQEISSSYTTTTTITAPPSGNEREKVKTVPLHLEEDIRPEMKEDIHDPTYQDEEGPPPKLEYVWRNIILMVLLHLGGLYGIILVPSCKLYTCLFGIFYYMTSALGITAGAHRLWSHRTYKARLPLRIFLIIANTMAFQNDVYDWARDHRAHHKFSETHADPHNSRRGFFFSHVGWLLVRKHPAVKEKGGKLDMSDLKAEKLVMFQRRYYKPGLLLMCFILPTLVPWYCWGETFVNSLFVSTFLRYTLVLNATWLVNSAAHLYGYRPYDKNIQSRENILVSLGAVGEGFHNYHHTFPFDYSASEYRWHINFTTFFIDCMAALGLAYDRKKVSKATVLARIKRTGDGSHKSS

SEQ ID NO: 2 - protein, 358 aa, Mus musculus
MPAHILQEISGAYSATTTITAPPSGGQQNGGEKFEKSSHHWGADVRPELKDDLYDPTYQDDEGPPPKLEYVWRNIILMALLHLGALYGITLVPSCKLYTCLFAYLYYVISALGITAGAHRLWSHRTYKARLPLRLFLIIANTMAFQNDVYEWARDHRAHHKFSETHADPHNSRRGFFFSHVGWLLVRKHPAVKEKGGKLDMSDLKAEKLVMFQRRYYKPDLLLMCFVLPTLVPWYCWGETFVNSLCVSTFLRYAVVLNATWLVNSAAHLYGYRPYDKNISSRENILVSMGAVGERFHNYHHAFPYDYSASEYRWHINFTTFFIDCMALLGLAYDRKRVSRAAVLARIKRTGDGSCKSG

SEQ ID NO: 3 - protein, 359 aa, Mus musculus
MPGHLLQEEMTPSYTTTTTITAPPSGSLQNGREKVKTVPLYLEEDIRPEMKEDIYDPTYQDEEGPPPKLEYVWRNIILMALLHVGALYGITLVPSCKLYTCLFAFVYYVISIEGIGAGAHRLWSHRTYKARLPLRIFLIIANTMAFQNDVYEWARDHRAHHKFSETHADPHNSRRGFFFSHVGWLLVRKHPAVKEKGGKLDMSDLKAEKLVMFQRRYYKPGILLMCFILPTLVPWYCWGETFLNSFYVATLLRYAVVLNATWLVNSAAHLYGYRPYDKNIDPRQNALVSLGSMGEGFHNYHHAFPYDYSASEYRWHINFTTFFIDCMAALGLAYDRKRVSKATVLARIKRTGDGSHKSG

SEQ ID NO: 4 - protein, 353 aa, Mus musculus
MTAHLPQEISSRCSTTNIMEPHSRRQQDGEEKMPLQAEDIRPEIKDDLYDPSYQDEEGPPPKLEYVWRNIIFMALLHVGALYGITLVPSCKVYTWLLGVFYNVYAGLGITAGAHRLWSHRTYKARLPLRIFLIMANTMAFQNDVYEWARDHRAHHKFSETHADPHNSRRGFFFSHVGWLLVRKHPAVKEKGKNLDMSDLKAEKLVMFQRRYYKLAVTLMFIILPTLVPWYLWGETFQHSLCVSNFLRYAVLLNFTWLVNSAAHLYGYRPYDRGIGARENPFVSMASLGEGFHNYHHTFPYDYSVSEYRWHINFTTFFIDCMAALGLAYDRKKVSKAVVLARIKRTGDGSHKSS

SEQ ID NO: 5 - protein, 359 aa, Homo sapiens
MPAHLLQDDISSSYTTTTTITAPPSRVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYFWGETFQNSVFVATFLRYAVVLNATWLVNSAAHLFGYRPYDKNISPRENILVSLGAVGEGFHNYHHSFPYDYSASEYRWHINFTTFFIDCMAALGLAYDRKKVSKAAILARIKRTGDGNYKSG

SEQ ID NO: 6 - protein, 330 aa, Homo sapiens
MPGPATDAGKIPFCDAKEEIRAGLESSEGGGGPERPGARGQRQNIVWRNVVLMSLLHLGAVYSLVLIPKAKPLTLLWAYFCFLLAALGVTAGAHRLWSHRSYRAKLPLRIFLAVANSMAFQNDIFERSRDHRAHHKYSETDADPHNARRGFFFSHIGWLFVRKHRDVIEKGRKLDVTDLLADPVVRIQRKYYKISVVLMCFVVPTLVPWYIWGESLWNSYFLASILRYTISLNISWLVNSAAHMYGNRPYDKHISPRQNPLVALGAIGEGFHNYHHTFPFDYSASEFGLNFNPTTWFIDFMCWLGLATDRKRATKPMIEARKARTGDSSA

SEQ ID NO: 7 - protein, 327 aa, Caenorhabditis elegans
MTQIKVDAIISKQFLAADLNEIRQMQEESKKQVIKMEIVWKNVALFVALHIGALVGLYQLVFQAKWATVGWVFLLHTLGSMGVTGGAHRLWAHRAYKATLSWRVFLMLINSIAFQNDIIDWARDHRCHHKWTDTDADPHSTNRGMFFAHMGWLLVKKHDQLKIQGGKLDLSDLYEDPVLMFQRKNYLPLVGIFCFALPTFIPVVLWGESAFIAFYTAALFRYCFTLHATWCINSVSHWVGWQPYDHQASSVDNLWTSIAAVGEGGHNYHHTFPQDYRTSEHAEFLNWTRVLIDFGASIGMVYDRKTTPEEVIQRQCKKFGCETEREK

SEQ ID NO: 8 - protein, 337 aa, Basidiomycte Lentinula edodes
MSDVVKKGSDDSSATVTSQVTDSPTIPDVDNYVAYTIKNTKALPPVTWSNLLNELNWLSVYILTIPPLVGFVGAFYVKLQWETAVWAVAYYFLTGLGITAGYHRLWAHRAFNASLPLQYVLAILGAGSLQGSIKWWSRGHRAHHRYTDTELDPYNAHKGFWFSHVGWMLVKPRRKPGVADVSDLRHNPVVKWQHKHYLSLILFMGFILPSIVAYVGWGDAKGGFIYAGVIRLVFVHHSTFCVNSLAHWLGETPFDDKHTPRDHMITAFVTIGEGYHNFHHQFPMDYRNAIKWYQYDPTKWTIWVLAKLGLASHLKVFPDNEVRKGQLTMELKKLRRT